(19) United States
(12) Patent Application Publication
(10) Pub. No.: US 2026/0232373 A1
(43) Pub. Date: Aug. 13, 2026

(54) CONTROL OF PACING PULSES PROVIDED TO A PATIENT

(71) Applicant: Medtronic, Inc., Minneapolis, MN (US)

(72) Inventors: Lars M. Mattison, Blaine, MN (US); Lynn A. Davenport, New Brighton, MN (US); Alexander J. Hill, Blaine, MN (US); Heather Lainee Mattson OConnell, Maple Grove, MN (US); Jeffrey P. Bade, Scottsdale, AZ (US); Ben W. Herberg, Andover, MN (US); Daniel C. Sigg, Saint Paul, MN (US)

(21) Appl. No.: 19/156,264

(22) PCT Filed: Jan. 23, 2024
(86) PCT No.: PCT/IB2024/050646

§ 371 Date: Aug. 13, 2025

Related U.S. Application Data
(60) Provisional application No. 63/484,569, filed on Feb. 13, 2023.

(51) Int. Cl. A61B 18/14; A61B 18/00

(52) U.S. Cl. CPC A61B 18/1492; A61B 2018/00357; A61B 2018/00577; A61B 2018/00702; A61B 2018/00839; A61B 2018/1407

(57) ABSTRACT

Methods and systems for controlling pacing pulses delivered to a patient are disclosed. One example method includes monitoring, after ablation of a heart of the patient is performed, an electrical signal that causes the heart of the patient to beat. The method may also include determining, with an electronic processor and based on the electrical signal, that an amount of time between consecutive cardiac cycles is greater than a desired value. The method may also include controlling, automatically and with the electronic processor, an electrode to deliver the pacing pulses to the heart in response to determining that the amount of time between consecutive cardiac cycles is greater than the desired value.

This application claims the benefit of U.S. Provisional Patent Application Ser. No. 63/484,569, filed Feb. 13, 2023, the entire content of which is incorporated herein by reference.

### FIELD

The present technology is generally related to methods and systems for controlling pacing pulses provided to a heart of a patient.

### BACKGROUND

There are many medical treatments that involve instances of cutting, ablating, coagulating, destroying, or otherwise changing the physiological properties of tissue. These techniques can be used beneficially to change the electrophysiological properties of tissue, such as those associated with cardiac arrhythmias or other electrophysiological abnormalities. An arrhythmia can take place in the atria, for example, as in atrial tachycardia, atrial fibrillation (“AF”), or atrial flutter. The arrhythmia can also take place in the ventricle, for example, as in ventricular tachycardia. Additionally, there may be ectopic sites within the heart that produce premature activations from such tissue sites, producing arrhythmogenic conduction patterns.

One approach to treating an arrhythmia includes creating one or more lesions that compartmentalize an aberrant pathway and direct electrical conduction along selected pathways to promote organized signal conduction, while also isolating AF triggers from connecting with the atria. Often, the application of energy is used to destroy cells at the ablation site while leaving the surrounding structures of the organ largely intact. Radiofrequency (“RF”) energy and cryogenic cooling have been found to be highly viable in this regard and are commonly employed. Other ablative techniques include the application of ultrasound, microwave, laser, cytotoxic agents, etc. Yet another ablative technique includes applying energy in the form of pulsed electrical fields (PEF).

Before, during, and/or after ablation, it may be desirable to attempt to ensure that the heart is beating within a range of desired heart rates.

### SUMMARY

Pulsed field ablation (PFA) is a term used to explain an application of energy in the form of PEF to ablate cardiac tissues (e.g., creating lesions) via mechanisms of electroporation. Electric fields and lesions created by the electric fields may be dependent on many factors including, but not limited to, applied voltage, electrode configuration, pulse wave form, number and length of pulse trains, modality of energy application (i.e. bipolar versus unipolar) and proximity of electrode to target tissue.

In some instances, after delivering (e.g., providing, applying, etc.) PFA energy to the heart of a patient, the heart of the patient may experience a pause (or multiple pauses) in which the heart rate of the patient slows down. For example, a pause (e.g., a vagal pause) may be a prolonged pause (e.g., longer than desired or expected) in between consecutive heartbeats/cardiac cycles of the heart. It is hypothesized that, in some situations, vagal pauses may be caused by electrical fields from the PFA energy that may momentarily stimulate the vagal nerve, which in turn, momentarily slows conduction velocity of the vagal nerve and results in a vagal pause and/or decreased heart rate (also referred as bradycardia which may include or result from prolonged pauses in between consecutive heartbeats/cardiac cycles of the heart). While vagal pauses and/or bradycardia may occur after delivery of PFA energy to the heart, vagal pauses and/or bradycardia may occur at any time and may be caused by one or more of a variety of other factors.

For the health and safety of a patient, it is desirable for the patient to maintain a steady heart rate (e.g., above a predetermined heart rate) without vagal pauses. Since cardiac output is dependent on the volume of blood ejected by the heart (i.e. stroke volume), but also the rate at which it is ejected (i.e. heart rate), heart rate is a critical factor in determining cardiac output. A clinically significant vagal pause and/or bradycardia can result in a significant decrease of cardiac output, resulting in decrease in blood pressure and perfusion to critical organs. Accordingly, it is desirable to respond quickly when a vagal pause and/or bradycardia is detected in order to reduce a length of time of a vagal pause, an amount of vagal pauses experienced by the patient, and/or a length of time that the heart is beating at a decreased heart rate.

The techniques of this disclosure generally relate to automatically controlling pacing pulses provided to the heart of a patient in response to determining that the amount of time between consecutive cardiac cycles (e.g., heart beats) is greater than a desired value. In some instances, determining that the amount of time between consecutive cardiac cycles is greater than the desired value includes determining that a next cardiac cycle expected to follow a previous cardiac cycle has not been detected for longer than a predetermined amount of time since the previous cardiac cycle was detected (e.g., detecting a vagal pause). Such automatic control of pacing pulses may be particularly useful after or during ablation of the heart. However, such control may be performed in other situations as well that do not necessarily involve ablation of the heart.

In one example, the present disclosure provides a method of controlling pacing pulses provided to a patient. The method may include monitoring, after ablation of a heart of the patient is performed, an electrical signal that causes the heart of the patient to beat. The method may further include determining, with an electronic processor and based on the electrical signal, that an amount of time between consecutive cardiac cycles is greater than a desired value. The method may also include controlling, automatically and with the electronic processor, an electrode to deliver the pacing pulses to the heart in response to determining that the amount of time between consecutive cardiac cycles is greater than the desired value.

In some aspects, the method also includes controlling, with the electronic processor, the electrode to deliver pulsed field ablation (PFA) energy to the heart prior to determining that the amount of time between consecutive cardiac cycles is greater than the desired value.

In some aspects, the electrode includes a first electrode or first pair of electrodes, and the method further includes controlling, with the electronic processor, a second electrode or second pair of electrodes to deliver pulsed field ablation (PFA) energy to the heart prior to determining that the amount of time between consecutive cardiac cycles is greater than the desired value. The second (pair of) electrode(s) may be located at a different location within the patient than the first electrode. In some aspects, the first (pair of) electrode(s) is included on a diagnostic catheter (e.g., a coronary sinus catheter), and the second (pair of) electrode(s) is included on an ablation catheter (e.g., a pulmonary vein ablation catheter (PVAC)).

In some aspects, the electrode includes a first electrode or a first pair of electrodes, and the method also includes continuing to monitor the electrical signal while the pacing pulses are being delivered to the heart. The method may further include determining, with the electronic processor and based on the electrical signal, that a second amount of time between later consecutive cardiac cycles remains greater than the desired value. The method may further include controlling, automatically and with the electronic processor, a second electrode or second pair of electrodes located at a different location within the patient than the first electrode to deliver second pacing pulses to the heart in response to determining that the second amount of time between later consecutive cardiac cycles remains greater than the desired value.

In some aspects, determining that the amount of time between consecutive cardiac cycles is greater than the desired value includes determining the amount of time between consecutive cardiac cycles by determining an RR interval between a first R-wave in a first cardiac cycle and a second R-wave in a second cardiac cycle.

In some aspects, determining that the amount of time between consecutive cardiac cycles is greater than the desired value includes determining that a next cardiac cycle expected to follow a previous cardiac cycle has not been detected for longer than a predetermined amount of time since the previous cardiac cycle was detected.

In some aspects, the desired value includes a predetermined value established independently of previous cardiac cycles of the heart of the patient.

In some aspects, the desired value includes a predetermined value established based on a predetermined increase in time compared to an average amount of time between consecutive cardiac cycles for a predetermined amount of previously monitored cardiac cycles.

In another example, the present disclosure provides a cardiac pacing device that may include an electrode or pair of electrodes configured to deliver pacing pulses to a heart of a patient. The cardiac pacing device may further include an electronic processor coupled to the electrode(s) to provide a control signal to the electrode. The electronic processor may be configured to monitor, after ablation of the heart of the patient is performed, an electrical signal that causes the heart of the patient to beat. The electronic processor may be further configured to determine, based on the electrical signal, that an amount of time between consecutive cardiac cycles is greater than a desired value. The electronic processor may be further configured to automatically control the electrode(s) to deliver the pacing pulses to the heart in response to determining that the amount of time between consecutive cardiac cycles is greater than the desired value.

In some aspects, the electronic processor may be further configured to control the electrode or pair of electrodes to deliver pulsed field ablation (PFA) energy to the heart prior to determining that the amount of time between consecutive cardiac cycles is greater than the desired value.

In some aspects, the electrode includes a first electrode or first pair of electrodes, and the electronic processor may be further configured to control a second electrode or second pair of electrodes to deliver pulsed field ablation (PFA) energy to the heart prior to determining that the amount of time between consecutive cardiac cycles is greater than the desired value. The second (pair of) electrode(s) may be located at a different location within the patient than the first (pair of) electrode(s).

In some aspects, the electrode includes a first electrode or first pair of electrodes, and the electronic processor may be further configured to continue to monitor the electrical signal while the pacing pulses are being delivered to the heart. The electronic processor may be further configured to determine, based on the electrical signal, that a second amount of time between later consecutive cardiac cycles remains greater than the desired value. The electronic processor may be further configured to automatically control a second electrode or second pair of electrodes located at a different location within the patient than the first (pair of) electrode(s) to deliver second pacing pulses to the heart in response to determining that the second amount of time between later consecutive cardiac cycles remains greater than the desired value.

In some aspects, the electronic processor may be configured to determine that the amount of time between consecutive cardiac cycles is greater than the desired value by determining that a next cardiac cycle expected to follow a previous cardiac cycle has not been detected for longer than a predetermined amount of time since the previous cardiac cycle was detected.

In another example, the present disclosure provides a method of controlling pacing pulses provided to a patient. The method may include delivering pulsed field ablation (PFA) energy to a heart of the patient. The method may further include monitoring, after delivering the PFA energy to the heart of the patient, an electrical signal that causes the heart of the patient to beat. The method may further include detecting, with an electronic processor and based on the electrical signal, bradycardia of the heart between consecutive cardiac cycles of the heart by determining that an amount of time between the consecutive cardiac cycles is greater than a desired value. The method may further include controlling, automatically and with the electronic processor, an electrode or pair of electrodes to deliver the pacing pulses to the heart in response to detecting the bradycardia by determining that the amount of time between consecutive cardiac cycles is greater than the desired value.

In some aspects, delivering the PFA energy to the heart of the patient includes delivering, via the (pair of) electrode(s), the PFA energy to the heart of the patient prior to determining that the amount of time between consecutive cardiac cycles is greater than the desired value.

In some aspects, the electrode includes a first electrode or first pair of electrodes, and the method may further include controlling, with the electronic processor, a second electrode or second pair of electrodes to deliver the PFA energy to the heart. The second (pair of) electrode(s) may be located at a different location within the patient than the first (pair of) electrode(s).

In some aspects, the first (pair of) electrode(s) is included on a diagnostic catheter (e.g., a coronary sinus catheter), and the second (pair of) electrode(s) is included on an ablation catheter.

In some aspects, the electrode includes a first electrode or first pair of electrodes, and the method may further include continuing to monitor the electrical signal while the pacing pulses are being delivered to the heart. The method may further include determining, with the electronic processor and based on the electrical signal, that a second amount of time between later consecutive cardiac cycles remains greater than the desired value. The method may further include controlling, automatically and with the electronic processor, a second electrode or second pair of electrodes located at a different location within the patient than the first electrode to deliver second pacing pulses to the heart in response to determining that the second amount of time between later consecutive cardiac cycles remains greater than the desired value.

In some aspects, determining that the amount of time between consecutive cardiac cycles is greater than the desired value includes determining that a next cardiac cycle expected to follow a previous cardiac cycle has not been detected for longer than a predetermined amount of time since the previous cardiac cycle was detected.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A shows an example ablation system including a pulsed-field ablation catheter having a distal circular electrode array portion according to one example.

FIG. 1B shows a more detailed view of an additional medical device with a linear electrode array portion included in the system of FIG. 1A according to one example.

FIG. 2 is a block diagram of the generator of the of the ablation system of FIG. 1A according to one example.

FIG. 3 illustrates a flowchart of a method performed by an electronic processor of the generator of FIG. 2 to control delivery of pacing pulses to a heart of a patient.

### DETAILED DESCRIPTION

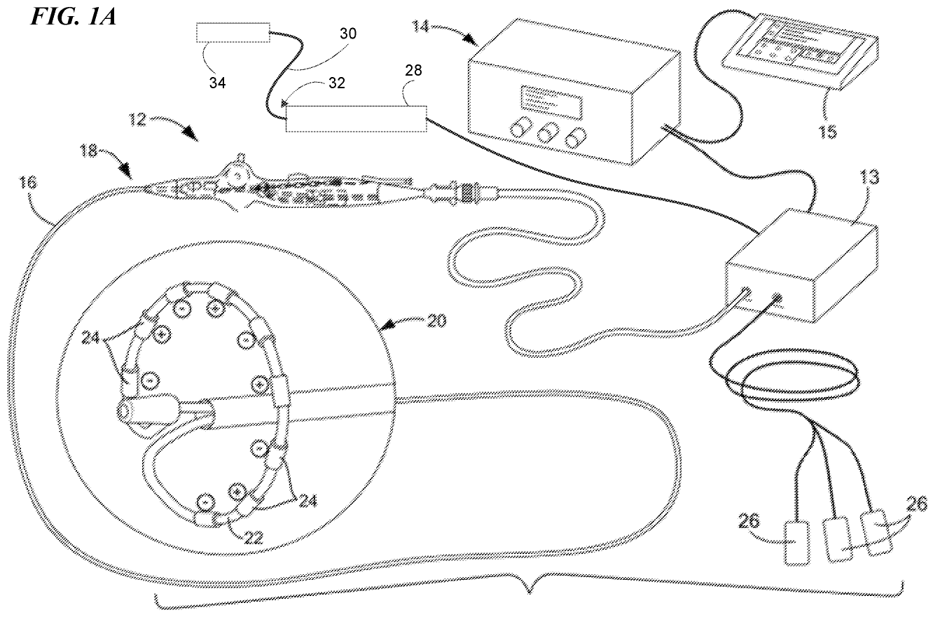

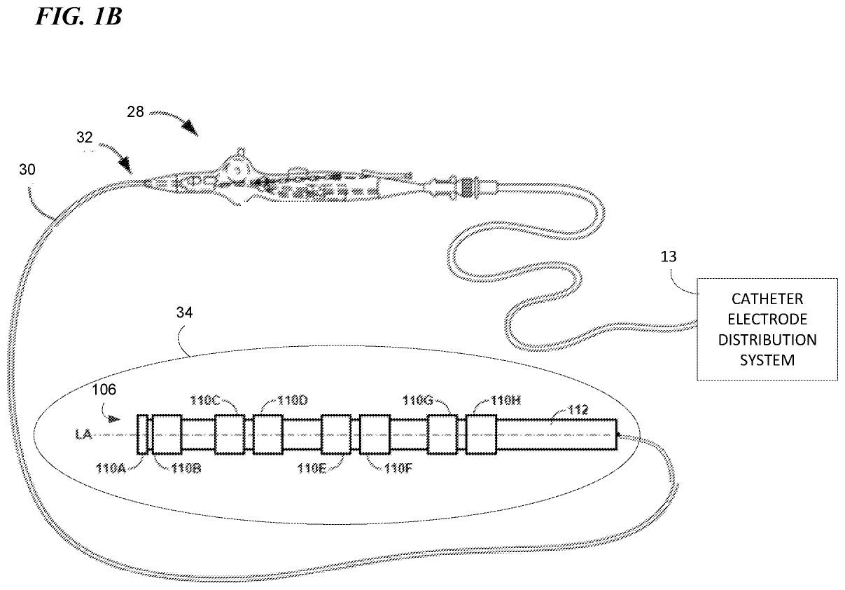

The present application provides, among other things, methods and systems for use during diagnosis and/or treatment of undesirable physiological or anatomical tissue regions, such as those contributing to aberrant electrical pathways in the heart. Referring now to the figures in which like reference designations refer to like elements, an example of a medical system constructed in accordance with principles of the present disclosure is shown in FIGS. 1A and 1B and generally designated as “10.” The system 10 generally includes a medical device 12 that may be coupled directly to an energy supply, for example, a pulse field ablation (PFA) generator 14 including an energy control, delivering system, and a monitoring system. In some aspects, the medical device 12 is coupled to the energy supply indirectly through a catheter electrode distribution system 13. A remote controller 15 may also be included in communication with the generator 14 for operating and controlling the various functions of the generator 14. The medical device 12 may generally include one or more diagnostic or treatment regions for energetic, therapeutic, and/or investigatory interaction between the medical device 12 and a treatment site. The treatment region(s) may deliver, for example, pulsed electroporation energy to a tissue area in proximity to the treatment region(s).

The medical device 12 may include an elongate body 16 passable through a patient's vasculature and/or positionable proximate to a tissue region for diagnosis or treatment, such as a catheter, sheath, or intravascular introducer. The elongate body 16 may define a proximal portion 18 and a distal portion 20, and may further include one or more lumens disposed within the elongate body 16 thereby providing mechanical, electrical, and/or fluid communication between the proximal portion 18 of the elongate body 16 and the distal portion 20 of the elongate body 16. The distal portion 20 may generally define the one or more treatment region(s) of the medical device 12 that are operable to monitor, diagnose, and/or treat a portion of a patient. The treatment region(s) may have a variety of configurations to facilitate such operation. In the case of purely bipolar pulsed field delivery, distal portion 20 includes electrodes that form the bipolar configuration for energy delivery. In an alternate configuration, a plurality of the electrodes 24 serve as one pole while a second device containing one or more electrodes (not pictured) would be placed to serve as the opposing pole of the bipolar configuration. For example, as shown in FIG. 1A, the distal portion 20 may include an electrode carrier arm 22 that is transitionable between a linear configuration and an expanded configuration in which the carrier arm 22 has an arcuate or substantially circular configuration. The carrier arm 22 may include the plurality of electrodes 24 (for example, nine electrodes 24, as shown in FIG. 1A) that are configured to deliver pulsed-field energy. Further, the carrier arm 22 when in the expanded configuration may lie in a plane that is substantially orthogonal to the longitudinal axis of the elongate body 16. The planar orientation of the expanded carrier arm 22 facilitates ease of placement of the plurality of electrodes 24 in contact with the target tissue. Alternatively, the medical device 12 may have a linear configuration with the plurality of electrodes 24. In one example, the distal portion 20 includes six electrodes 24 linearly disposed along a common longitudinal axis. In some instances, the distal portion/catheter 20 is referred to/used as a pulmonary vein isolation catheter or an ablation catheter.

The system 10 may further include three or more electrocardiogram (ECG) electrodes 26 configured to be placed in or on the patient and configured to be in communication with the generator 14 through the catheter electrode distribution box 13. The electrodes 26 may be used to monitor the patient's cardiac activity for use in determining how to control pacing pulses delivered to the heart of the patient (e.g., when/whether to deliver pacing pulses to the heart, a location/catheter to be used to deliver pacing pulses to the heart, etc.) as explained in greater detail below. For example, the electrodes 26 and/or other electrodes 24, 110 described herein may gather data that may be used by the generator 14 to determine QRS waves of the heartbeat/cardiac cycle of the patient. In some instances, based on consecutive QRS waves, the generator 14 determines, for example, an RR interval of the heart of the patient that is indicative of an instantaneous heart rate of the patient. In addition to monitoring, recording, or otherwise conveying measurements or conditions within the medical device 12 or the ambient environment at the distal portion 20 of the medical device 12, additional measurements may be made through connections to the multi-electrode catheter including for example temperature, electrode-tissue interface impedance, delivered charge, current, power, voltage, work, or the like in the generator 14 and/or the medical device 12. The surface ECG electrodes 26 may be in communication with the generator 14 for initiating or triggering one or more alerts, therapeutic deliveries, and/or pacing pulse deliveries during operation of the medical device 12. In some instances, additional or alternative information is used to monitor patient information such as cardiac cycle and timing. For example, the system 10 may receive intracardiac electrogram (EGM) information and/or other information from one or more other electrodes and/or devices/sensors. Additional neutral electrode patient ground patches (not pictured) may be employed to evaluate the desired bipolar electrical path impedance, as well as monitor and alert the operator upon detection of inappropriate and/or unsafe conditions, which include, for example, improper (either excessive or inadequate) delivery of charge, current, power, voltage and work performed by the plurality of electrodes 24; improper and/or excessive temperatures of the plurality of electrodes 24; improper electrode-tissue interface impedances; improper and/or inadvertent electrical connection to the patient prior to delivery of high voltage energy by delivering one or more low voltage test pulses to evaluate the integrity of the tissue electrical path.

The generator 14 may include an electrical current or pulse generator having a plurality of output channels, with each channel coupled to an individual electrode of the plurality of electrodes 24 or multiple electrodes of the plurality of electrodes 24 of the medical device 12. In some instances, the generator 14 is operable in one or more modes of operation, including for example: (i) bipolar energy delivery between at least two electrodes 24 or electrically-conductive portions of the medical device 12 within a patient's body, (ii) monopolar or unipolar energy delivery to one or more of the electrodes 24 or electrically-conductive portions on the medical device 12 within a patient's body and through either a second device within the body (not shown) or a patient return or ground electrode (not shown) spaced apart from the plurality of electrodes 24 of the medical device 12, such as on a patient's skin or on an auxiliary device positioned within the patient away from the medical device 12, for example, and (iii) a combination of the monopolar and bipolar modes.

The generator 14 provides electrical pulses to the medical device 12 to perform an electroporation procedure to cardiac tissue or other tissues within the body, for example, renal tissue, airway tissue, and organs or tissue within the cardiothoracic space. “Electroporation” utilizes high amplitude pulses to effectuate a physiological modification (e.g., permeabilization) of the cells to which the energy is applied. Such pulses may preferably be short (e.g., nanosecond, microsecond, or millisecond pulse width) in order to allow application of high voltage, high current (for example, 20 or more amps) without long duration of electrical current flow that results in significant tissue heating and muscle stimulation. Preferably, the pulsed energy induces the formation of microscopic pores or openings in the cell membrane. Depending upon the characteristics of the electrical pulses, an electroporated cell can survive electroporation (e.g., “reversible electroporation”) or die (e.g., irreversible electroporation, “IEP”). Reversible electroporation may be used to transfer agents, including large molecules, into targeted cells for various purposes, including alteration of the action potentials of cardiac myocytes.

In some instances, the generator 14 is configured (e.g., programmed) to deliver pulsed, high voltage electric fields appropriate for achieving desired pulsed, high voltage ablation (or pulsed field ablation). As a point of reference, the pulsed, high voltage, non-radiofrequency, ablation effects of the present disclosure are distinguishable from DC current ablation, as well as thermally-induced ablation attendant with conventional RF techniques. In some instances, the pulse trains delivered by generator 14 are delivered at a frequency less than 3 kHz, and in an example configuration, 1 kHz, which is a lower frequency than radiofrequency treatments. The pulsed-field energy in accordance with the present disclosure is sufficient to induce cell death for purposes of completely blocking an aberrant conductive pathway along or through cardiac tissue, destroying the ability of the so-ablated cardiac tissue to propagate or conduct cardiac depolarization waveforms and associated electrical signals.

In some instances, the plurality of electrodes 24 perform diagnostic functions such as collection of intracardiac electrograms (EGM) and selective pacing of intracardiac sites for diagnostic purposes. In one configuration, the measured ECG signals are transferred from the catheter electrode energy distribution system 13 to an electrophysiology (EP) recording system input box (not shown) that is included with generator 14. The plurality of electrodes 24 may also monitor the proximity to target tissues and quality of contact with such tissues using impedance based measurements with connections to the catheter electrode energy distribution system 13. The catheter electrode energy distribution system 13 may include high speed relays to disconnect/reconnect specific electrode 24 from the generator 14 during therapies. Immediately following the pulsed energy deliveries, the relays reconnect the electrodes 24 so they may be used for diagnostic purposes.

While one or more of the electrodes 24 may perform both pacing (e.g., provide pacing pulses) and ablation in some situations, in some instances, the system 10 may include one or more optional additional medical devices and associated elongated structures/catheters that may be configured to perform pacing. In one example, the system 10 includes another instance of the medical device 12 with another instance of the distal portion 20. In another example, the system 10 includes one or more additional medical devices 28 with an elongate body 30 including a proximal portion 32 and a distal portion 34 that is different than the distal portion 20 as shown in FIGS. 1A and 1B. These components 28, 30, 32, and 34 that share a name with previously-described components 12, 16, 18, and 20, respectively, may be similar to and function similarly to their respective like-named components except for the differences described below.

In some instances, the distal portion 34 (which is shown in enlarged scale in FIG. 1B) includes catheter/elongated structure 112 carrying a plurality of electrodes 110A-110H (collectively, “electrodes 110”). Catheter 112 may include a distal portion 106 and a proximal portion 108. Electrodes 110 may be generally positioned at distal portion 106, while proximal portion 108 may be ultimately connected to the catheter electrode distribution system 13. Similar to the electrodes 24 described previously herein, the electrodes 110 may be configured to deliver pacing pulses and/or to perform diagnostic functions such as collection of intracardiac electrograms (EGM). In some instances, unlike the electrodes 24 described previously herein, the electrodes 110 are not used to deliver PFA energy to the heart. In some instances, the electrodes 110 are dedicated pacing and/or diagnostic electrodes 110, and the catheter 112 is a dedicated pacing and/or diagnostic catheter 112. In some instances, the catheter 112 is referred to/used as a diagnostic catheter, e.g., a coronary sinus catheter.

In some instances, use of multiple electrodes 24, 110 and/or catheters 20, 112 allows pacing pulses to be delivered to the heart at a different location than a location where PFA energy is delivered to the heart as explained in greater detail below. In some instances, use of multiple electrodes 24, 110 and/or catheters 20, 112 allows pacing pulses to be delivered to the heart at a plurality of different locations simultaneously or sequentially as explained in greater detail below.

The electrodes 24 and/or 110 may be of any suitable geometry. Example geometries of electrodes include, but are not necessarily limited to, circular (e.g., ring) electrodes surrounding the body of the lead, conformable electrodes, cuff electrodes, segmented electrodes (e.g., electrodes disposed at different circumferential positions around the lead instead of a continuous ring electrode), or a combination thereof (e.g., ring electrodes and segmented electrodes). Electrodes 110 may be axially distributed along longitudinal axis LA of the catheter 112. The catheter 112 and the electrodes 24, 110 shown in FIGS. 1A and 1B are merely examples. In some instances, the catheter 112 includes more or fewer electrodes 110, and/or the distal portion 20 may include more or fewer electrodes 24. Additionally or alternatively, the electrodes 24 and/or 110 may be arranged in different configurations including using coils or other return electrodes. In some instances, the catheter 112 and/or the distal portion 20 have a different shape at a point where the catheter 112 and/or the distal portion 20 contacts tissue of the patient.

In some aspects, the plurality of electrodes 24 deliver therapeutic biphasic pulses having a preprogrammed pattern and duty cycle as explained in U.S. Pat. No. 10,531,914 (U.S. application Ser. No. 15/228,406), which is incorporated by reference and appended herein. In some aspects, a pulse train when delivered from a bipolar electrode array (such as the array shown in FIG. 1) produces lesions in cardiac muscle in the range of approximately 2-3 millimeters deep, 4-7 millimeters deep, and/or the like. Increased voltage may correspondingly increase the lesion depth.

As explained previously herein, the system 10 may include ECG electrodes 26 electrically couplable to the generator 14 and configured to measure electrical signals from the heart. The ECG measurements, or Einthoven signals, made by the ECG electrodes 26 may be sequentially or simultaneously made with the delivery of the pulse trains from the plurality of electrodes 24. In an example configuration, three ECG electrodes 26 are adhered the surface of the patient and are further coupled to the generator 14. The generator 14 may be configured to process and correlate the measured Einthoven signals in order to determine when to deliver pulses of PFA energy and/or whether and when to deliver pacing pulses. In some instances, the generator 14 is configured to process and correlate the measured Einthoven signals in order to determine whether and when to deliver pacing pulses to the heart as explained in greater detail below. For example, the generator 14 may be programmed with predetermined measured patient parameters, for example, timing parameters associated with a desirable heart rate value or related value to control timing of the delivery of pacing pulses to the heart of the patient as explained in greater detail below. When at least one of the predetermined measured patient parameters are met, the generator 14 may initiate the delivery of pacing pulses.

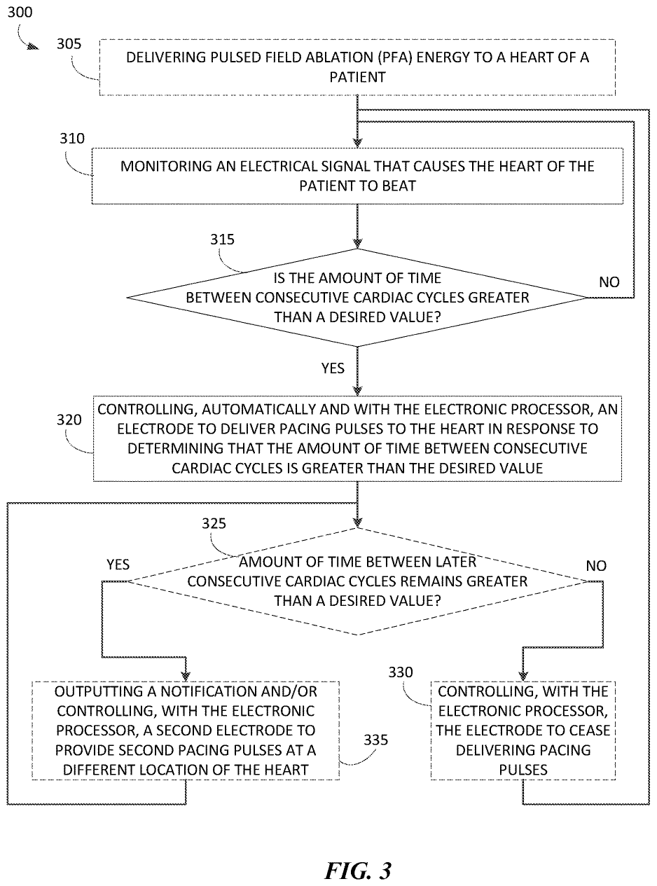

In some instances, the generator 14 automatically controls timing of delivery of pacing pulses to the heart of the patient in response to determining that an amount of time between consecutive cardiac cycles (e.g., heart beats) is greater than a desired value. For example, in some instances, the generator 14 may perform a method 300 shown in FIG. 3 to control delivery of pacing pulses to the heart of the patient. In some instances, determining that the amount of time between consecutive cardiac cycles is greater than the desired value includes determining that a next cardiac cycle expected to follow a previous cardiac cycle has not been detected for longer than a predetermined amount of time since the previous cardiac cycle was detected (e.g., detecting a vagal pause). Such automatic control of pacing pulses may be particular useful after or during ablation of the heart. However, such control may be performed in other situations as well that do not necessarily involve ablation of the heart.

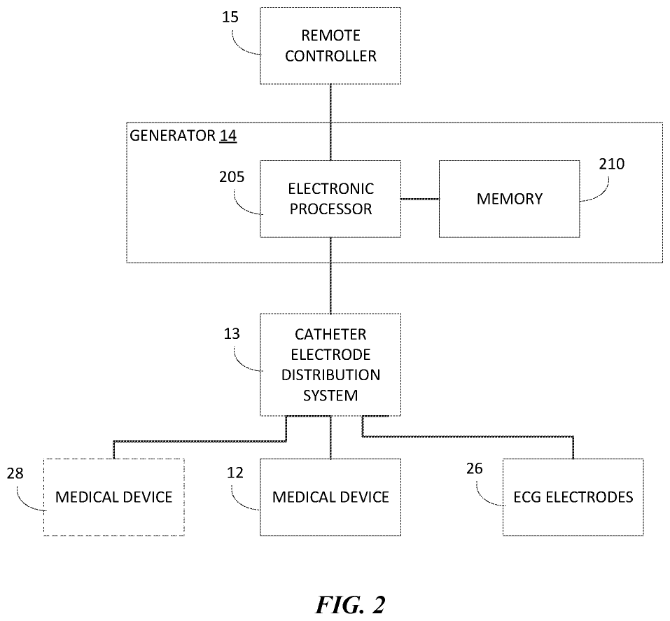

FIG. 2 is a block diagram of the generator 14 of the ablation system 10 according to one example. In the example shown, the generator 14 includes an electronic processor 205 (for example, a microprocessor or another electronic device). The electronic processor 205 may be electrically connected to a memory 210 and may include input and output interfaces to couple with other devices of the system 10, for example, the remote controller 15 and the catheter electrode distribution system 13 as shown in FIG. 2.

The memory 210 may include read only memory (ROM), random access memory (RAM), other non-transitory computer-readable media, or a combination thereof. The electronic processor 205 is configured to receive instructions and data from the memory 210 and execute, among other things, the instructions. In particular, the electronic processor 205 executes instructions or algorithms stored in the memory 210 to provide for the automated operation and performance of the features, sequences, calculations, or procedures described herein.

In some aspects, the generator 14 includes fewer or additional components in configurations different from that illustrated in FIG. 2. For example, the generator 14 may include a display and/or an integrated user input device in addition to or as an alternative to the remote controller 15. As another example, in some aspects, the generator 14 includes one or more additional electronic processors that may perform specific functions and that are communicatively coupled (electrically or electromagnetically) to each other and/or to the electronic processor 205. When an electronic processor 205 is referred to herein, it should be understood that the functionality being performed by the electronic processor 205 may be performed by one or more electronic processors 205 within the generator 14 and/or distributed within other devices of the system 10.

Other devices of the system 10 may include similar components as the generator 14. For example, the catheter electrode distribution system 13, the remote controller 15, and/or the medical devices 12, 28 may each include an electronic processor and/or a memory similar to those described previously herein with respect to the generator 14. In some aspects, these other devices 12, 13, 15, 28 may additionally or alternatively have other components that allow each device 12 ,13, 15, 28 to perform its respective functionality as described herein.

In FIG. 2, the medical device 28 is shown in dashed lines to indicate that the medical device 28 may not be included in the system 100 in some instances. Even though other devices are shown in solid lines in FIG. 2, in some instances, some of such devices may not be included in the system 100.

In some instances, the medical device 12 includes a catheter 20 that provides both PFA energy for ablation (e.g., a first signal/pulse train) and pacing pulses for pacing the heart of the patient (e.g., a second signal/pulse train that is different from the first signal/pulse train). For example, the catheter 20 provides the PFA energy and the pacing pulses at different times and using the same or different electrodes 24 as explained herein. In some instances, one or more of the electrodes 24 may also perform diagnostics (e.g., EGM monitoring/recording). In some instances, the optional medical device(s) 28 includes a catheter 112 configured to provide only pacing pulses, configured to provide only diagnostic capabilities, or configured to provide both pacing pulses and diagnostic capabilities.

In some instances, the electronic processor 205 of the generator 14 is configured to act as a PFA generator/controller, a pacing controller, and/or a diagnostic controller. In some instances, the electronic processor 205 automatically controls timing of delivery of pacing pulses to the heart of the patient in response to determining that an amount of time between consecutive cardiac cycles (e.g., heart beats) is greater than a desired value. For example, in some instances, the generator 14 may perform a method 300 shown in FIG. 3 to control delivery of pacing pulses to the heart of the patient. As explained previously herein, such automatic control of delivery of pacing pulses to the heart addresses a technological problem (e.g., a patient experiencing vagal pauses or a decreased heart rate/bradycardia, for example, caused by delivery of PFA energy) by responding quickly when a vagal pause and/or bradycardia is detected. The quick and automatic response provided by the method 300 reduces a length of time of a vagal pause, an amount of vagal pauses experienced by the patient, and/or a length of time that the heart is beating at a decreased heart rate. Accordingly, the quick and automatic response by the system 100 results in increased health and safety of the patient by attempting to maintain a steady heart rate (e.g., above a predetermined heart rate) without vagal pauses.

FIG. 3 illustrates a flowchart of a method 300 performed by the electronic processor 205 of the generator 14 (in conjunction with other devices in the system 100 in some instances) to control delivery of pacing pulses to the heart of the patient. While a particular order of processing steps is indicated in FIG. 3 as an example, timing and ordering of such steps may vary where appropriate without negating the purpose and advantages of the examples set forth herein. In FIG. 3, some blocks/steps are shown in dashed lines to indicate that these blocks/steps are optional and may not be performed in some instances of the method 300. Even though other blocks/steps are shown in solid lines in FIG. 3, in some instances, some of such blocks/steps may not be included in the method 300.

At block 305, pulsed field ablation (PFA) energy is optionally delivered to a heart of a patient. In instances where block 305 is performed, the method 300 may be performed in conjunction with application of PFA energy to control delivery of pacing pulses to the heart during and/or after PFA energy delivery. However, as indicated previously herein, the method 300 may also be performed in instances where PFA energy is not being delivered to the heart. In some instances, the PFA energy may be delivered to the heart using one or more electrodes of the catheter/distal portion 20 of FIG. 1A or using one or more electrodes of the catheter/distal portion 112 of FIG. 1B.

At block 310, an electrical signal that causes a heart of a patient to beat is monitored. For example, an electrocardiogram (ECG) of the heart of the patient is determined by the electronic processor 205 of the generator 14. In some aspects, the ECG may be determined by another electronic processor of another device. The ECG is determined based on an electrical signal received from one or more electrodes. The electrodes that the provide the electrical signal that allows for the ECG to be determined may include one or more of the electrodes 24, one or more of the electrodes 110, one or more of the ECG electrodes 26, or a combination thereof. In some aspects, a first electrode 24 or 110 that delivers PFA energy to a treatment site of the heart is also used to monitor the electrical signal of the heart that is used to generate the ECG. Though a single electrode is discussed herein for purposes of discussion for electrode 24 and electrode 110, it is to be understood that pairs of electrodes are contemplated and within the scope of this disclosure regarding electrode 24 and electrode 100 unless discussed otherwise. In one example, unipolar signals are measured from an indwelling PFA catheter with an electrode 24 or 110, and PFA energy is delivered from the same electrode 24, 110. As another example, bipolar signals may be measured from an indwelling PFA catheter from two electrodes 24 or 110, and PFA energy may be delivered in bipolar fashion from both electrodes 24 or 110. In some instances, the two above-noted examples may be mixed and matched. In one example, unipolar signals are measured by an indwelling catheter when bipolar PFA energy is delivered to the treatment site or vice versa. In some aspects, a second electrode (e.g., ECG electrode 26) that is separate from the electrodes 24, 110 and that is not used to deliver PFA energy to the treatment site is used to monitor the electrical signal of the heart that is used to generate the ECG. In some instances, one or more of the electrodes 24 may be used to deliver PFA energy to the heart, and one or more of the electrodes 110 may be used to monitor the cardiac signal or vice versa. In some instances, the electronic processor 205 receives additional or alternative information to monitor a cardiac cycle. For example, the electronic processor 205 may receive intracardiac electrogram (EGM) information and/or other information from one or more other electrodes and/or devices/sensors. In instances of the method 300 in which block 305 is performed to deliver PFA energy to the heart of the patient, monitoring of the electrical signal that causes the heart of the patient to beat may occur after ablation of the heart of the patient is performed.

At block 315, the electronic processor 205 of the generator 14 determines, based on the electrical signal monitored at block 310, whether an amount of time between consecutive cardiac cycles is greater than a desired value (e.g., whether bradycardia is detected). In some instances, the electronic processor 205 is configured to determine the amount of time between consecutive cardiac cycles by determining a first time interval between occurrences of a first wave in a current cardiac cycle and a second wave included in the electrical signal (e.g., ECG) of one or more previous cardiac cycles. In some aspects, the first wave and the second wave are successive occurrences of the same first type of wave included in the electrical signal. In other words, in some aspects, the electronic processor 205 is configured to determine a first time interval between successive occurrences of a first type of wave (e.g., an R-wave, a P-wave, a Q-wave, etc.) included in the electrical signal. For example, the electronic processor 205 is configured to determine an RR interval between a first R-wave in a first cardiac cycle and a second R-wave in a second cardiac cycle (e.g., an RR interval between consecutive heartbeats/cardiac cycles). Other types of waves and intervals may also be used in some aspects.

In some instances, the electronic processor 205 is configured to determine that the amount of time between consecutive cardiac cycles is greater than the desired value by determining that a next cardiac cycle expected to follow a previous cardiac cycle has not been detected for longer than a predetermined amount of time since the previous cardiac cycle was detected. For example, if the next cardiac cycle has not occurred within a certain time period, then the electronic processor 205 determines that the amount of time between cardiac cycles is greater than desired even though the next cardiac cycle has not yet occurred/been detected. Accordingly, the electronic processor 205 may determine that the amount of time between consecutive cardiac cycles is greater than the desired value even though a second/next cardiac cycle has not yet occurred/been detected. Such a configuration allows the electronic processor 205 to detect a pause (e.g., a vagal pause) in which the heart of the patient does not beat/engage in a cardiac cycle for a longer time period than desired/expected. In some instances, detection of a vagal pause occurs when the next heartbeat/cardiac cycle is not detected for the predetermined amount of time or when the next heartbeat/cardiac cycle is detected but occurs more than the predetermined amount of time after the previous cardiac heartbeat/cardiac cycle. In other words, in some instances, at block 315, the electronic processor 205 is configured to detect, based on the electrical signal, a vagal pause of the heart between consecutive cardiac cycles of the heart by determining that the amount of time between the consecutive cardiac cycles is greater than the desired value.

In some instances, the desired value at block 315 includes a predetermined value (e.g., a predetermined amount of time). In some instances, the desired value includes a predetermined value established independently of previous cardiac cycles of the heart of the patient. For example, the predetermined value may be established to initiate/trigger pacing pulses to be delivered in response to the amount of time between consecutive cardiac cycles being greater than a cutoff threshold that is undesirable for most or all patients regardless of their historical heartbeat/cardiac cycle timing/patterns. For example, the predetermined value may be set to 40 beats per minute (BPM), 45 BPM, or the like.

In some instances, the desired value at block 315 includes a predetermined value established based on a predetermined increase in time compared to an average amount of time between consecutive cardiac cycles for a predetermined amount of previously monitored cardiac cycles of the patient. Using such a predetermined value may allow the electronic processor 205 to initiate/trigger pacing pulses to be delivered in response to determining an increase (e.g., percentage increase of time between heartbeats/cardiac cycles) compared to previously monitored cardiac cycles of the patient that causes pacing to be triggered. For example, the predetermined value may be a 30% increase of time between heartbeats/cardiac cycles (or a corresponding 30% decrease in heart rate), a 50% increase of time between heartbeats/cardiac cycles (or a corresponding 50% decrease in heart rate), or the like. In such instances, the heart rate of the patient may be above the cutoff threshold explained in the previous example, but the electronic processor 205 may nevertheless initiate/trigger pacing pulses to be delivered to the heart based on the decrease in heart rate being greater than the predetermined value. Accordingly, in some instances, the electronic processor 205 may determine whether the amount of time between consecutive cardiac cycles is greater than the desired value (at block 315) using either one of the predetermined values explained in the two above examples. In other words, the electronic processor 205 may initiate/trigger pacing pulses (at block 320) in response to either one of the monitored characteristics explained in the two above examples falling outside a range of it respective predetermined value.

In some instances, the electronic processor 205 is configured to determine that the amount of time between consecutive cardiac cycles is greater than the desired value by determining that bradycardia has persisted for a predetermined time period. For example, if bradycardia and/or pauses are detected for two seconds but then the heart's activity returns to normal functioning, pacing may not be initiated. However, if bradycardia and/or pauses persist for a predetermined time period, pacing may be initiated. In some instances, the predetermined time period may be set to correspond to a time period expected to cause syncope (e.g., loss of consciousness in a non-sedated/anesthetized patient). Accordingly, in such instances, if bradycardia and/or pauses persist for longer than the predetermined time period (e.g., approximately 6-10 seconds or the like), pacing may be initiated. In some instances, the electronic processor 205 is configured to determine that the amount of time between consecutive cardiac cycles is greater than the desired value by determining that an average heart rate of the patient over the predetermined time period is below a threshold value even though some of the specific amounts of time between two specific consecutive cardiac cycles within the predetermined time period may not be greater than the desired value. For example, pacing may be initiated in response to determining that an average heart rate of the patient has been below a threshold value (e.g., 30 BPM, 40 BPM, or the like) for a predetermined time period (e.g., five seconds, 6-10 seconds, or the like). Similarly, in some instances, the electronic processor 205 is configured to determine that the amount of time between consecutive cardiac cycles is greater than the desired value by determining that an amount of cardiac cycles within a predetermined period is less than a desired amount even though some of the specific amounts of time between two specific consecutive cardiac cycles within the predetermined time period may not be greater than the desired value. In some instances, pacing may be initiated/triggered in response to detecting pauses and/or bradycardia in other manners. For example, pauses and/or bradycardia may be detected in other manners as disclosed in U.S. Pat. No. 11,260,234 (U.S. application Ser. No. 16/702,928) and/or U.S. Pat. No. 9,937,352 (U.S. application Ser. No. 14/920,228), the entire contents of each of which are hereby incorporated by reference and appended herein.

At block 315, when the electronic processor 205 determines that the amount of time between consecutive cardiac cycles is not greater than the desired value, the method 300 proceeds back to block 310 to continue monitoring the electrical signal that causes the heart of the patient to beat. On the other hand, at block 315, when the electronic processor 205 determines that the amount of time between consecutive cardiac cycles is greater than the desired value, the method 300 proceeds to block 320.

At block 320, the electronic processor 205 automatically controls an electrode 24, 110 to deliver pacing pulses to the heart in response to determining that the amount of time between consecutive cardiac cycles is greater than the desired value. As indicated by the previous explanation of block 315, in some instances, the electronic processor 205 automatically controls an electrode 24, 110 to deliver the pacing pulses to the heart in response to detecting a vagal pause by determining that the amount of time between consecutive cardiac cycles is greater than the desired value. The pacing pulses may be delivered at a predetermined rate (e.g., 70 BPM, or the like).

In some instances, the electrode 24, 110 used to deliver pacing pulses to the heart (at block 320) is the same electrode 24, 110 that is used to deliver PFA energy to the heart (at block 305) prior to determining that the amount of time between consecutive cardiac cycles is greater than the desired value. In such instances, the PFA energy and the pacing pulses may be delivered to the same location of the heart. In some instances, the same catheter 20, 112 may be used to deliver pacing pulses to the heart (at block 320) and to deliver PFA energy to the heart (at block 305), but different electrodes 24, 110 of the catheter 20, 112 may be used to separately deliver the pacing pulses and the PFA energy. In such instances, the PFA energy and the pacing pulses may be delivered to the same area of the heart but at slightly different locations corresponding to the locations of the separate electrodes 24, 110.

In some instances, a first catheter 112 including a first electrode 110 is used to deliver the pacing pulses (at block 320) and a second catheter 20 including a second electrode 24 is used to deliver the PFA energy (at block 305). Accordingly, the catheters 112 and 20 (and their electrodes 110 and 24, respectively) may be located at different locations within the patient to deliver pacing pulses to a different area of the heart than where the PFA energy is delivered. For example, the first catheter 112 may be a diagnostic catheter (e.g., a coronary sinus catheter) located at or near a diagnostic location, e.g., near a coronary sinus of the heart, and the second catheter 20 may be an ablation catheter located at or near a pulmonary vein of the heart. As another example, the first catheter 112 (or another catheter) configured to deliver pacing pulses (at block 320) may be located at a left ventricle (e.g., epicardial left ventricle) and/or a right ventricle (e.g., epicardial right ventricle).

In some instances, when different electrodes 24, 110 and/or different catheters 20, 112 are used to deliver the pacing pulses and PFA energy, the pacing pulses and PFA energy may be delivered in an overlapping time period (e.g., at the same time) and/or at different time periods.

After performing block 320 and while the pacing pulses are being delivered to the heart of the patient, the electronic processor 205 may continue to monitor the electrical signal that causes the heart of the patient to beat in a similar manner as described previously herein with respect to block 310. At block 325, the electronic processor 205 determines, based on the electrical signal of the patient that is being monitored, whether a second amount of time between later consecutive cardiac cycles remains greater than the desired value. In some instances, the determination(s) made at block 325 is similar to the determination(s) made at block 315.

At block 325, when the second amount of time between later consecutive cardiac cycles does not remain greater than the desired value (e.g., the heart rate of the patient has increased to a desired rate), the method 300 proceeds to block 330. In such situations, the control of the pacing pulses performed in a quick and automatic manner (at block 320) may result increased health and safety to the patient since their heart rate is restored to a desired rate very quickly. At block 330, the electronic processor 205 may control the electrode 24, 110 that is providing pacing pulses to cease providing the pacing pulses. In some of such instances, at block 330, the electronic processor 205 may control the electrode 24, 110 that is providing pacing pulses to continue providing pacing pulses for a limited time period (e.g., five seconds, ten seconds, or the like) before controlling the electrode 24, 110 to cease providing the pacing pulses. In some instances, after performing block 330, the method 300 proceeds back to block 310 (or block 305) to continue monitoring the electrical signal that causes the heart of the patient to beat.

On the other hand, at block 325, when the second amount of time between later consecutive cardiac cycles remains greater than the desired value (or when the second amount of time does not at least begin to shorten/decrease), the method 300 proceeds to block 335. At block 335, the electronic processor 205 may automatically control, a second electrode 24, 110 located at a different location within the patient than the first electrode 24, 110 that is already delivering first pacing pulses to the heart to deliver additional or alternative pacing pulses (e.g., second pacing pulses) to the heart in response to determining that the second amount of time between later consecutive cardiac cycles remains greater than the desired value. In other words, because first pacing pulses already being delivered by a first electrode 24, 110 have not increased the heart rate of the patient somewhat or to a desired heart rate, the system 100 may implement additional or alternative pacing at a different location of the heart (e.g., using a different second electrode 24, 110 and/or catheter 20, 112).

For example, if pacing pulses being delivered by an ablation catheter 20 to a pulmonary vein of the heart are not causing the heart rate of the patient to increase somewhat or to a desired rate, the electronic processor 205 may automatically control a diagnostic catheter 112 (e.g., a coronary sinus catheter) to deliver additional or alternative pacing pulses to a region such as the coronary sinus. As another example, if pacing pulses being delivered by a diagnostic catheter 112 to region such as a coronary sinus of the heart are not causing the heart rate of the patient to increase somewhat or to a desired rate, the electronic processor 205 may automatically control an ablation catheter 20 to deliver additional or alternative pacing pulses to a pulmonary vein of the heart. As yet another example, if pacing pulses being delivered by a diagnostic catheter 112 to the coronary sinus of the heart are not causing the heart rate of the patient to increase to a desired rate, the electronic processor 205 may automatically control an additional electrode and/or catheter (e.g., a third catheter) located at a different location (e.g., a third location) of the heart to deliver additional or alternative pacing pulses to the different location of the heart. For example, the third location may include a ventricular chamber such as a left ventricle (e.g., epicardial left ventricle) and/or a right ventricle (e.g., epicardial right ventricle). As indicated by the “additional or alternative” language in the previous examples, at block 335, the electronic processor 205 may control the initial/first pacing pulses to continue to be delivered while second pacing pulses (and/or third pacing pulses) are also delivered to a different area of the heart or may control the initial/first pacing pulses to cease to be delivered while the second pacing pulses (ad/or third placing pulses) are delivered to the different area of the heart.

As indicated in FIG. 3, at block 335, the electronic processor 205 may additionally or alternatively output a notification (e.g., an audible or visual notification on the remote controller 15) to a user of the system 100. The notification may indicate that the second amount of time between later consecutive cardiac cycles remains greater than the desired value so that the user can take additional action if desired. In some instances, after performing block 335, the method 300 proceeds back to block 325 to continue monitoring the electrical signal of the heart of the patient to determine whether the amount of time between even later consecutive cardiac cycles remains greater than the desired value. In some instances, the electronic processor 205 may execute blocks 325 and 335 multiple times to provide pacing pulses from different electrodes 24, 110 and/or catheters 20, 112 until the heart rate of the patient begins to improve (e.g., increase) toward a desired rate and/or improves (e.g., increases) to the desired rate. In other words, the electronic processor 205 may automatically control different electrodes 24, 110 and/or catheters 20, 112 to provide pacing pulses in response to determining that pacing pulses being provided from a first electrode 24, 110 and/or catheter 20, 112 are not improving the patient's condition (e.g., not decreasing the amount of time between consecutive heartbeats/cardiac cycles). Such control being performed in a quick and automatic manner may result increased health and safety to the patient since their heart rate is restored to a desired rate very quickly.

The ranges included herein (e.g., the percentage ranges of the first time interval) are examples. One or both ends of each of these example ranges may vary by, for example, 1%, 5%, 10%, etc. These example ranges are intended to delineate an approximate time range during which a myocardium/heart wall thickness of the treatment site is estimated/expected to be low or at a minimum thickness compared to the myocardium/heart wall thickness at other times in a cardiac cycle.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Example 1. A method of controlling pacing pulses provided to a patient, the method comprising: monitoring, after ablation of a heart of the patient is performed, an electrical signal that causes the heart of the patient to beat; determining, with an electronic processor and based on the electrical signal, that an amount of time between consecutive cardiac cycles is greater than a desired value; and controlling, automatically and with the electronic processor, an electrode or a pair of electrodes to deliver the pacing pulses to the heart in response to determining that the amount of time between consecutive cardiac cycles is greater than the desired value.

Example 2. The method of Example 1, further comprising controlling, with the electronic processor, the electrode or pair of electrodes to deliver pulsed field ablation (PFA) energy to the heart prior to determining that the amount of time between consecutive cardiac cycles is greater than the desired value.

Example 3. The method of either Example 1 or Example 2, wherein the electrode includes a first electrode or a first pair of electrodes, and further comprising controlling, with the electronic processor, a second electrode or second pair of electrodes to deliver pulsed field ablation (PFA) energy to the heart prior to determining that the amount of time between consecutive cardiac cycles is greater than the desired value, wherein the second (pair of) electrode(s) is located at a different location within the patient than the first (pair of) electrode(s).

Example 4. The method of Example 3, wherein the first (pair of) electrode(s) is included on a diagnostic catheter such as a coronary sinus catheter, and wherein the second (pair of) electrode(s) is included on an ablation catheter.

Example 5. The method of any of Examples 1-4, wherein the electrode includes a first electrode or first pair of electrodes, and further comprising: continuing to monitor the electrical signal while the pacing pulses are being delivered to the heart; determining, with the electronic processor and based on the electrical signal, that a second amount of time between later consecutive cardiac cycles remains greater than the desired value; and controlling, automatically and with the electronic processor, a second electrode or second pair of electrodes located at a different location within the patient than the first (pair of) electrode(s) to deliver second pacing pulses to the heart in response to determining that the second amount of time between later consecutive cardiac cycles remains greater than the desired value.

Example 6. The method of any of Examples 1-5, wherein determining that the amount of time between consecutive cardiac cycles is greater than the desired value includes determining the amount of time between consecutive cardiac cycles by determining an RR interval between a first R-wave in a first cardiac cycle and a second R-wave in a second cardiac cycle.

Example 7. The method of any of Examples 1-5, wherein determining that the amount of time between consecutive cardiac cycles is greater than the desired value includes determining that a next cardiac cycle expected to follow a previous cardiac cycle has not been detected for longer than a predetermined amount of time since the previous cardiac cycle was detected.

Example 8. The method of any of Examples 1-7, wherein the desired value includes a predetermined value established independently of previous cardiac cycles of the heart of the patient.

Example 9. The method of any of Examples 1-7, wherein the desired value includes a predetermined value established based on a predetermined increase in time compared to an average amount of time between consecutive cardiac cycles for a predetermined amount of previously monitored cardiac cycles.

Example 10. A cardiac pacing device comprising: an electrode or pair of electrodes configured to deliver pacing pulses to a heart of a patient; and an electronic processor coupled to the electrode to provide a control signal to the electrode(s), the electronic processor configured to monitor, after ablation of the heart of the patient is performed, an electrical signal that causes the heart of the patient to beat, determine, based on the electrical signal, that an amount of time between consecutive cardiac cycles is greater than a desired value, and automatically control the electrode to deliver the pacing pulses to the heart in response to determining that the amount of time between consecutive cardiac cycles is greater than the desired value.

Example 11. The cardiac pacing device of Example 10, wherein the electronic processor is further configured to control the electrode(s) to deliver pulsed field ablation (PFA) energy to the heart prior to determining that the amount of time between consecutive cardiac cycles is greater than the desired value.

Example 12. The cardiac pacing device of either Example 10 or Example 11, wherein the electrode includes a first electrode or first pair of electrodes, and wherein the electronic processor is further configured to control a second electrode or second pair of electrodes to deliver pulsed field ablation (PFA) energy to the heart prior to determining that the amount of time between consecutive cardiac cycles is greater than the desired value, wherein the second (pair of) electrode(s) is located at a different location within the patient than the first electrode.

Example 13. The cardiac pacing device of either Example 10 or Example 11, wherein the electrode includes a first electrode or first pair of electrodes, and wherein the electronic processor is further configured to: continue to monitor the electrical signal while the pacing pulses are being delivered to the heart; determine, based on the electrical signal, that a second amount of time between later consecutive cardiac cycles remains greater than the desired value; and automatically control a second electrode or second pair of electrodes located at a different location within the patient than the first (pair of) electrode(s) to deliver second pacing pulses to the heart in response to determining that the second amount of time between later consecutive cardiac cycles remains greater than the desired value.

Example 14. The cardiac pacing device of any of Examples 10-14, wherein the electronic processor is configured to determine that the amount of time between consecutive cardiac cycles is greater than the desired value by determining that a next cardiac cycle expected to follow a previous cardiac cycle has not been detected for longer than a predetermined amount of time since the previous cardiac cycle was detected.

Example 15. A method of controlling pacing pulses provided to a patient, the method comprising: delivering pulsed field ablation (PFA) energy to a heart of the patient; monitoring, after delivering the PFA energy to the heart of the patient, an electrical signal that causes the heart of the patient to beat; detecting, with an electronic processor and based on the electrical signal, bradycardia of the heart between consecutive cardiac cycles of the heart by determining that an amount of time between the consecutive cardiac cycles is greater than a desired value; and controlling, automatically and with the electronic processor, an electrode or pair of electrodes to deliver the pacing pulses to the heart in response to detecting the bradycardia by determining that the amount of time between consecutive cardiac cycles is greater than the desired value.

Example 16. The method of Example 15, wherein delivering the PFA energy to the heart of the patient includes delivering, via the electrode(s), the PFA energy to the heart of the patient prior to determining that the amount of time between consecutive cardiac cycles is greater than the desired value.

Example 17. The method of either Example 15 of Example 16, wherein the electrode includes a first electrode or first pair of electrodes, and further comprising controlling, with the electronic processor, a second electrode or second pair of electrodes to deliver the PFA energy to the heart, wherein the second (pair of) electrode(s) is located at a different location within the patient than the first (pair of) electrode(s).

Example 18. The method of Example 17, wherein the first (pair of) electrode(s) is included on a diagnostic catheter such as a coronary sinus catheter, and wherein the second (pair of) electrode(s) is included on an ablation catheter.

Example 19. The method of any of Examples 15-18, wherein the electrode includes a first electrode or first pair of electrodes, and further comprising: continuing to monitor the electrical signal while the pacing pulses are being delivered to the heart; determining, with the electronic processor and based on the electrical signal, that a second amount of time between later consecutive cardiac cycles remains greater than the desired value; and controlling, automatically and with the electronic processor, a second electrode or second pair of electrodes located at a different location within the patient than the first (pair of) electrode(s) to deliver second pacing pulses to the heart in response to determining that the second amount of time between later consecutive cardiac cycles remains greater than the desired value.

Example 20. The method of any of Examples 15-19, wherein determining that the amount of time between consecutive cardiac cycles is greater than the desired value includes determining that a next cardiac cycle expected to follow a previous cardiac cycle has not been detected for longer than a predetermined amount of time since the previous cardiac cycle was detected.

What is claimed is:

1. A cardiac pacing device comprising:
an electrode configured to deliver pacing pulses to a heart of a patient; and
an electronic processor coupled to the electrode to provide a control signal to the electrode, the electronic processor configured to
monitor, after ablation of the heart of the patient is performed, an electrical signal that causes the heart of the patient to beat,
determine, based on the electrical signal, that an amount of time between consecutive cardiac cycles is greater than a desired value, and
automatically control the electrode to deliver the pacing pulses to the heart in response to determining that the amount of time between consecutive cardiac cycles is greater than the desired value.

2. The cardiac pacing device of claim 1, wherein the electronic processor is further configured to control the electrode to deliver pulsed field ablation (PFA) energy to the heart prior to determining that the amount of time between consecutive cardiac cycles is greater than the desired value.

3. The cardiac pacing device of claim 1, wherein the electrode includes a first electrode, and wherein the electronic processor is further configured to control a second electrode to deliver pulsed field ablation (PFA) energy to the heart prior to determining that the amount of time between consecutive cardiac cycles is greater than the desired value, wherein the second electrode is located at a different location within the patient than the first electrode.

4. The cardiac pacing device of claim 3, further comprising a diagnostic catheter and an ablation catheter, wherein the first electrode is included on the diagnostic catheter, and wherein the second electrode is included on the ablation catheter.

5. The cardiac pacing device of claim 1, wherein the electrode includes a first electrode, and wherein the electronic processor is further configured to:
continue to monitor the electrical signal while the pacing pulses are being delivered to the heart;
determine, based on the electrical signal, that a second amount of time between later consecutive cardiac cycles remains greater than the desired value; and
automatically control a second electrode located at a different location within the patient than the first electrode to deliver second pacing pulses to the heart in response to determining that the second amount of time between later consecutive cardiac cycles remains greater than the desired value.

6. The cardiac pacing device of claim 1, wherein the electronic processor is configured to determine that the amount of time between consecutive cardiac cycles is greater than the desired value by determining that a next cardiac cycle expected to follow a previous cardiac cycle has not been detected for longer than a predetermined amount of time since the previous cardiac cycle was detected.

7. The cardiac pacing device of claim 1, wherein the electronic processor is configured to determine that the amount of time between consecutive cardiac cycles is greater than the desired value includes determining the amount of time between consecutive cardiac cycles by determining an RR interval between a first R-wave in a first cardiac cycle and a second R-wave in a second cardiac cycle.

8. The cardiac pacing device of claim 1, wherein the desired value includes a predetermined value established independently of previous cardiac cycles of the heart of the patient.

9. The cardiac pacing device of-any claim 1, wherein the desired value includes a predetermined value established based on a predetermined increase in time compared to an average amount of time between consecutive cardiac cycles for a predetermined amount of previously monitored cardiac cycles.
